(19) United States
(12) Patent Application Publication
(10) Pub. No.: US 2022/0246259 A1
(43) Pub. Date: Aug. 4, 2022

(54) LIFESTYLE IMPROVEMENT SPECIFIC MEASURE PRESENTING DEVICE, LIFESTYLE IMPROVEMENT SPECIFIC MEASURE PRESENTING METHOD, RECORDING MEDIUM, AND LIFESTYLE IMPROVEMENT SPECIFIC MEASURE PRESENTING SYSTEM

(71) Applicant: NEC Solution Innovators, Ltd., Koto-ku, Tokyo (JP)

(72) Inventors: Hirofumi TANAKA, Tokyo (JP); Toshikazu TAJIRI, Tokyo (JP); Tohru MIYAZAKI, Tokyo (JP)

(73) Assignee: NEC Solution Innovators, Ltd., Koto-ku, Tokyo (JP)

(21) Appl. No.: 17/605,270

(22) PCT Filed: Apr. 23, 2020
(86) PCT No.: PCT/JP2020/017481

§ 371 Date: Oct. 21, 2021

(30) Foreign Application Priority Data
Apr. 26, 2019 (JP) 2019-084865

(51) Int. Cl. G16H 20/00; G16H 10/20

(52) U.S. Cl. CPC G16H 20/00; G16H 10/20

(57) ABSTRACT

A lifestyle improvement specific measure presenting device includes a formulating unit which formulates a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

### TECHNICAL FIELD

The present invention relates to a lifestyle improvement specific measure presenting device, a lifestyle improvement specific measure presenting method, a recording medium, and a lifestyle improvement specific measure presenting system.

### BACKGROUND ART

In Japan, where the birthrate is declining and the population is aging, extending the healthy life expectancy of the population while reducing the nation's overall increasing national health care costs is an important issue.

In response to the government's “Japan Revitalization Strategy,” the guidelines for health services were partially revised in March 2014. Based on the revision, for example, it is assumed that health service providers will formulate and implement implementation plans for health services such as data health plans in order to implement effective and efficient health services in accordance with the PDCA (Plan-Do-Check-Action) cycle using health and medical information.

In other words, instead of implementing projects blindly, insurance providers are required to enhance the effectiveness of their projects by using data and taking a scientific approach.

It is generally believed that it is not easy to make individuals more interested in their own health and to make them review their lifestyle habit. Patent Literature (PTL) 1 describes a health condition predicting device that can predict test values for medical examinations using such as AI (Artificial Intelligence) analysis technology, which is a means of utilizing data, and make individuals aware of the need to improve their lifestyle habit. The health condition predicting device described in PTL 1 supports individuals in their efforts to change their behavior toward “prevention of ill health” and “health promotion”.

In addition, an information presentation sheet has been developed that predicts the future health condition of an individual using a health checkup result prediction model derived from accumulated past regular health checkup data. The prediction of specific future images by the health condition predicting device and the information presentation sheet described in PTL 1 can motivate individuals to improve their lifestyle and participate in health guidance.

In addition, PTL 2 describes a health management server that provides an online health guidance service that allows for comprehensibility and continuity of use by sending written advice messages to health management targets based on the results of dietary analysis, etc.

### CITATION LIST

### Patent Literature

PTL 1: International Publication No. WO 2017/204233
PTL 2: Japanese Patent No. 6083661

### SUMMARY OF INVENTION

### Technical Problem

In general, immediately after a regular medical checkup or health guidance, the motivation of health management targets to improve their lifestyle is high. However, as time goes by, the motivation of many health management targets declines.

If specific ways to improve one's lifestyle are also presented together to the health management target at the time when the future health condition is predicted, the rate of initiation of lifestyle improvement by the health management target is likely to increase. However, when lifestyle improvement methods are presented, if the methods, which are not related to the lifestyle improvement strategy of the health management target, are presented, the motivation of the health management target will decrease.

In other words, if a method which is not related to the lifestyle improvement strategy of the health management target is presented, the problem arises that the lifestyle improvement method will not be implemented continuously. The above-mentioned lifestyle improvement strategy is the behavioral strategy of the health management target to improve his/her lifestyle habit.

The health management server described in PTL 2 sends advice messages along with health management guidance based on the diet and health condition of the health management target. However, the content of the health management guidance is unilaterally defined by experts.

Therefore, the advice message sent by the health management server described in PTL 2 may not be related to the lifestyle improvement strategy of the health management target. If an advice message that is not related to the lifestyle improvement strategy is sent, it is expected that the motivation of the health management target will decrease. Also, PTL 1 does not describe any means to be able to solve the above problem.

### Object of Invention

Therefore, it is an object of the present invention to provide a lifestyle improvement specific measure presenting device, a lifestyle improvement specific measure presenting method, a recording medium, and a lifestyle improvement specific measure presenting system that can present specific measures for lifestyle improvement which users can work on with high motivation, solving the above-mentioned problems.

### Solution to Problem

A lifestyle improvement specific measure presenting device according to the present invention includes: a formulating means which formulates a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

A lifestyle improvement specific measure presenting method according to the present invention includes: formulating a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

A computer-readable recording medium recording a lifestyle improvement specific measure presenting program according to the present invention, when executed by a computer, stores the lifestyle improvement specific measure presenting program causing the computer to execute: formulating a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

A lifestyle improvement specific measure presenting system according to the present invention includes: a predicting device which predicts a user's future health condition; and a lifestyle improvement specific measure presenting device which includes a formulating means which formulates a lifestyle improvement strategy, which is a behavioral strategy for the user for improving a predetermined lifestyle habit of the user, on the basis of information, which is a predicted result output by the predicting device, indicating the predetermined lifestyle habit, included in the predicted result of the future health condition of the user if the predetermined lifestyle habit is improved.

### Advantageous Effects of Invention

According to this invention, it is possible to present specific measures for lifestyle improvement which users can work on with high motivation.

### BRIEF DESCRIPTION OF DRAWINGS

FIG. 1 is an explanatory diagram showing an example of a health checkup result prediction simulation system according to the present invention.

FIG. 2 is a block diagram showing an example of the configuration of the first example embodiment of a lifestyle improvement specific measure presenting device according to the present invention.

FIG. 3 is an explanatory diagram showing an example of lifestyle improvement strategy information accumulated in a lifestyle improvement strategy accumulating unit 102.

FIG. 4 is an explanatory diagram showing an example of lifestyle improvement relevance information accumulated in a lifestyle improvement relevance accumulating unit 104.

FIG. 5 is an explanatory diagram showing an example of lifestyle improvement specific measure information accumulated in a lifestyle improvement specific measure accumulating unit 105.

FIG. 6 is an explanatory diagram showing an example of displaying a list of the lifestyle improvement specific measure information.

FIG. 7 is an explanatory diagram showing an example of displaying of one lifestyle improvement specific measure information.

FIG. 8 is a flowchart showing the operation of the lifestyle improvement strategy formulation process by a lifestyle improvement specific measure presenting device 100 of the first example embodiment.

FIG. 9 is a flowchart showing the operation of the specific measures for lifestyle improvement presentation process by the lifestyle improvement specific measure presenting device 100 of the first example embodiment.

FIG. 10 is an explanatory diagram showing an example of a hardware configuration of the lifestyle improvement specific measure presenting device according to the present invention.

FIG. 11 is a block diagram showing an overview of the lifestyle improvement specific measure presenting device according to the present invention.

FIG. 12 is a block diagram showing an overview of a lifestyle improvement specific measure presenting system according to the present invention.

### DESCRIPTION OF EMBODIMENTS

[Description of Configuration]

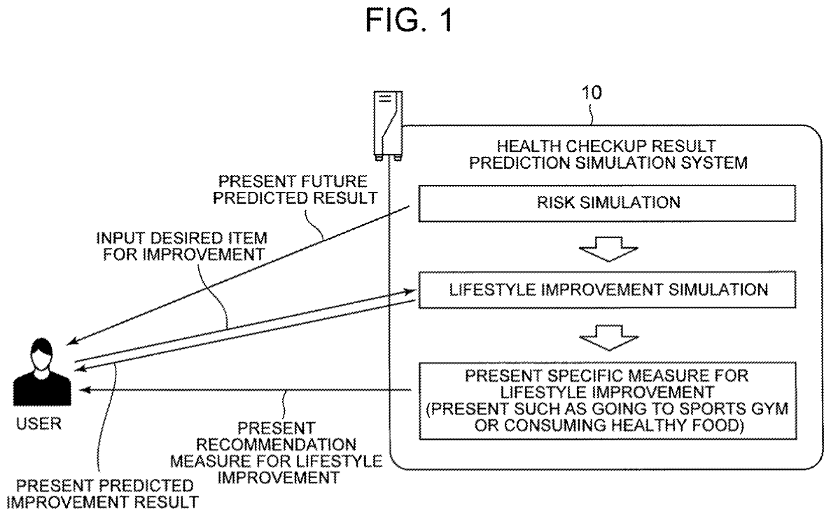

An example embodiment of the present invention will be described below with reference to the drawings. FIG. 1 is an explanatory diagram showing an example of a health checkup result prediction simulation system according to the present invention.

The health checkup result prediction simulation system 10 shown in FIG. 1 is a system that predicts the future health checkup results based on the user's health checkup results for the past two years by simulating the results if the user continues his or her current lifestyle or if the user reviews his or her current lifestyle, respectively.

The user shown in FIG. 1 is, for example, the health management target described above. The amount of health checkup results used by the health checkup result prediction simulation system 10 is not limited to the past two years.

As shown in FIG. 1, the health checkup result prediction simulation system 10 presents the predicted future health checkup results to the user. The user inputs the desired items for improvement to the health checkup result prediction simulation system 10 based on the presented future health checkup results. Then, the health checkup result prediction simulation system 10 performs a lifestyle improvement simulation using the desired items for improvement that have been input.

Next, the health checkup result prediction simulation system 10 presents the user with the predicted improvement results obtained from the lifestyle improvement simulation, i.e., the future health checkup results when the lifestyle is reviewed. In addition, the health checkup result prediction simulation system 10 determines the user's lifestyle improvement strategy based on the predicted improvement results.

Next, the health checkup result prediction simulation system 10 presents the user with specific measures for lifestyle improvement, which are specific measures for improving lifestyle habits, such as going to a sports gym or consuming healthy food, according to the established lifestyle improvement strategy. The lifestyle improvement specific measure presenting device that presents specific measures for lifestyle improvement in the health checkup result prediction simulation system 10 shown in FIG. 1 is a device that solves the above problem.

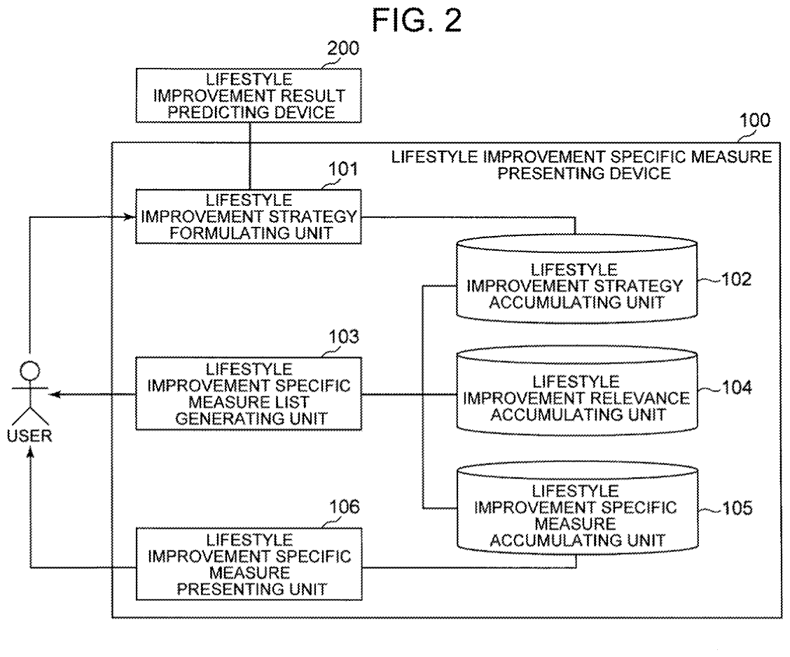

FIG. 2 is a block diagram showing an example of the configuration of the first example embodiment of a lifestyle improvement specific measure presenting device according to the present invention. As shown in FIG. 2, the lifestyle improvement specific measure presenting device 100 of this example embodiment has a lifestyle improvement strategy formulating unit 101, a lifestyle improvement strategy accumulating unit 102, a lifestyle improvement specific measure list generating unit 103, a lifestyle improvement relevance accumulating unit 104, a lifestyle improvement specific measure accumulating unit 105, and a lifestyle improvement specific measure presenting unit 106.

As shown in FIG. 2, the lifestyle improvement specific measure presenting device 100 is communicatively connected to the lifestyle improvement result predicting device 200. The lifestyle improvement result predicting device 200 is a device that performs the lifestyle improvement simulation shown in FIG. 1. The lifestyle improvement result predicting device 200 is a health condition predicting device described in PTL 1, for example.

The lifestyle improvement strategy formulating unit 101 formulates a lifestyle improvement strategy for the user based on the predicted results of the future health condition in the case that the user's lifestyle habit is improved, which are acquired from the lifestyle improvement result predicting device 200.

When the lifestyle improvement result predicting device 200 is the health condition predicting device described in PTL 1, the lifestyle improvement strategy formulating unit 101 may use the types of lifestyle habits input by the user to the health condition predicting device that predicts future test values in formulating the lifestyle improvement strategy. The inputted lifestyle habits are, for example, the frequency of light sweaty exercise of 30 minutes or more per time, and the number of meals per day.

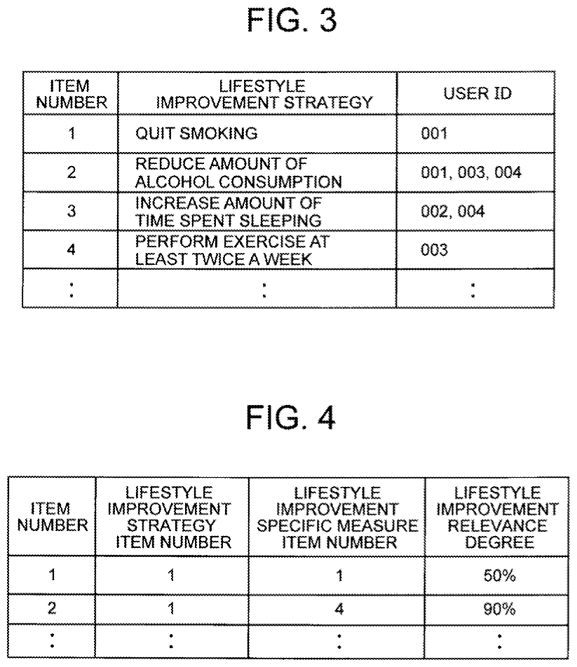

FIG. 3 is an explanatory diagram showing an example of lifestyle improvement strategy information accumulated in the lifestyle improvement strategy accumulating unit 102. The lifestyle improvement strategy information shown in FIG. 3 consists of an item number, a lifestyle improvement strategy, and a user ID. The item number is a number that uniquely identifies the lifestyle improvement strategy information.

The lifestyle improvement strategy is a behavioral strategy corresponding to the type of lifestyle habit that is input into the lifestyle improvement result predicting device 200 when, for example, future test values are predicted. FIG. 3 shows “quit smoking,” “reduce the amount of alcohol consumption,” “increase the amount of time spent sleeping,” “perform exercise at least twice a week,” etc. as lifestyle improvement strategies.

The user ID is a number that uniquely identifies the user who wishes to improve his or her lifestyle habit according to the lifestyle improvement strategy.

The predicted result of the future health condition acquired by the lifestyle improvement strategy formulating unit 101 includes the type of lifestyle habits that the user has input into the lifestyle improvement result predicting device 200. The lifestyle improvement strategy formulating unit 101 acquires one or more lifestyle improvement strategy information including lifestyle improvement strategies corresponding to the types of lifestyle habits included in the predicted results of the future health condition from the lifestyle improvement strategy accumulating unit 102.

Next, the lifestyle improvement strategy formulating unit 101 presents the acquired lifestyle improvement strategy information to the user. The user selects a lifestyle improvement strategy to be implemented by him/herself among one or more lifestyle improvement strategies indicated by the presented lifestyle improvement strategy information. The lifestyle improvement strategy formulating unit 101 records the user ID of the user who selected the lifestyle improvement strategy in the corresponding lifestyle improvement strategy information.

The lifestyle improvement specific measure list generating unit 103 acquires the lifestyle improvement relevance information corresponding to the lifestyle improvement strategy from the lifestyle improvement relevance accumulating unit 104. The lifestyle improvement specific measure list generating unit 103 acquires the lifestyle improvement relevance information corresponding to the lifestyle improvement strategy indicated by the lifestyle improvement strategy information in which the user ID of the user is recorded.

FIG. 4 is an explanatory diagram showing an example of lifestyle improvement relevance information accumulated in the lifestyle improvement relevance accumulating unit 104. The lifestyle improvement relevance information shown in FIG. 4 consists of an item number, a lifestyle improvement strategy item number, a lifestyle improvement specific measure item number, and a lifestyle improvement relevance degree. The item number is a number that uniquely identifies the lifestyle improvement relevance information.

The lifestyle improvement strategy item number is the item number of the lifestyle improvement strategy information. The lifestyle improvement specific measure item number is the item number of the lifestyle improvement specific measure information that is related to the lifestyle improvement strategy information indicated by the lifestyle improvement strategy item number included in the same lifestyle improvement relevance information.

The lifestyle improvement relevance degree is the degree to which the lifestyle improvement strategy information indicated by the lifestyle improvement strategy item number and the lifestyle improvement specific measure information indicated by the lifestyle improvement specific measure item number included in the same lifestyle improvement relevance information are related.

The administrator who operates the lifestyle improvement specific measure presenting device 100 may manually set the lifestyle improvement relevance degree when registering lifestyle improvement relevance information and lifestyle improvement specific measure information. The lifestyle relevance degree may be a value computed by AI that has learned the relationship between the lifestyle improvement strategy and the specific measures for lifestyle improvement.

The lifestyle improvement relevance information is registered in advance in the lifestyle improvement relevance accumulating unit 104 by the operator of the health checkup result prediction simulation system 10, for example. In addition, the lifestyle improvement specific measure list generating unit 103 may register the lifestyle improvement relevance information in the lifestyle improvement relevance accumulating unit 104.

Next, the lifestyle improvement specific measure list generating unit 103 acquires the lifestyle improvement specific measure information corresponding to the acquired lifestyle improvement relevance information from the lifestyle improvement specific measure accumulating unit 105.

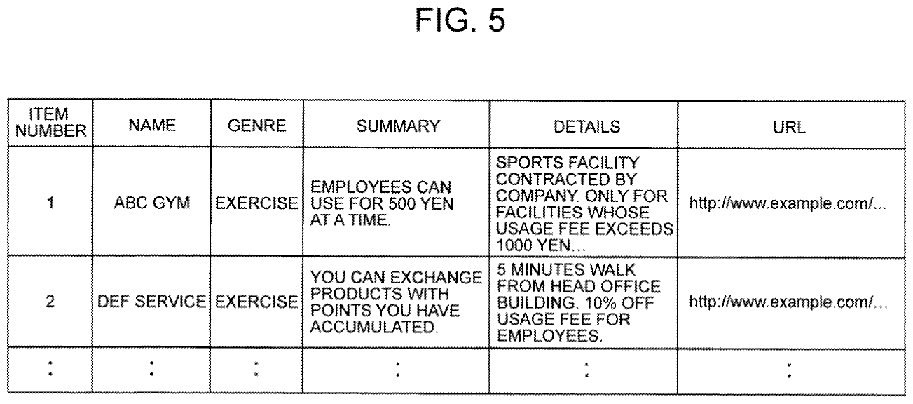

FIG. 5 is an explanatory diagram showing an example of lifestyle improvement specific measure information accumulated in the lifestyle improvement specific measure accumulating unit 105. The lifestyle improvement specific measure information shown in FIG. 5 consists of the item number, name, genre, summary, details, and URL (Uniform Resource Locator). The item number is a number that uniquely identifies the lifestyle improvement specific measure information.

The name, genre, summary, and details are information about the specific measures for lifestyle improvement, respectively. The URL is, for example, the URL of the home page of the company that provides the specific measures for lifestyle improving.

For example, the lifestyle improvement specific measure information is registered in advance in the lifestyle improvement specific measure accumulating unit 105 by the operator of the health checkup result prediction simulation system 10. In addition, the lifestyle improvement specific measure list generating unit 103 may register the lifestyle improvement specific measure information in the lifestyle improvement specific measure accumulating unit 105.

The lifestyle improvement specific measure list generating unit 103 presents the list of the acquired lifestyle improvement specific measure information to the user. When presenting the list of the lifestyle improvement specific measure information to the user, the lifestyle improvement specific measure list generating unit 103 may display the lifestyle improvement specific measure information in the order of the lifestyle improvement relevance degree set in the lifestyle relevance information.

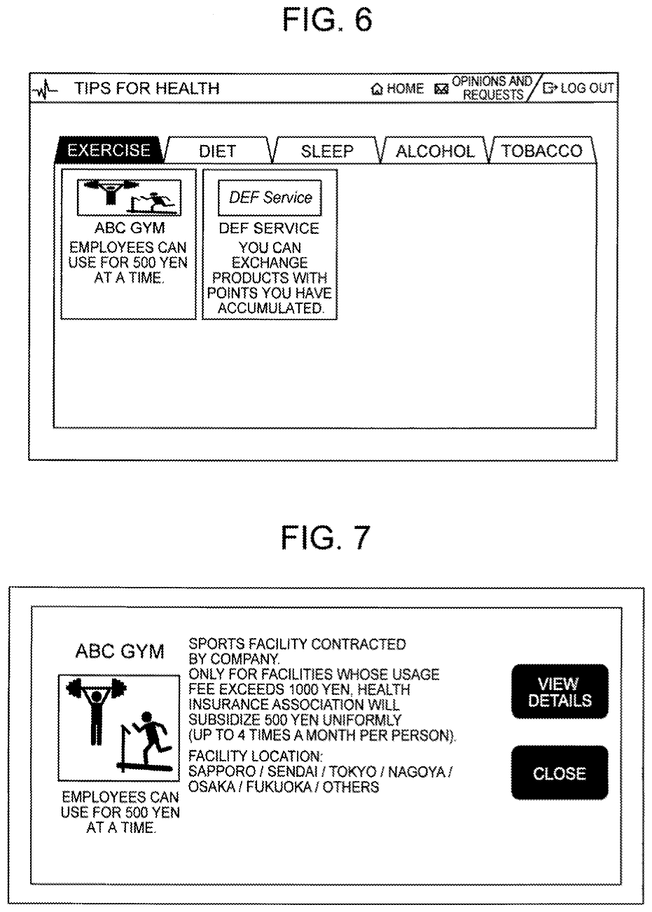

FIG. 6 is an explanatory diagram showing an example of displaying a list of the lifestyle improvement specific measure information. FIG. 6 shows an example of a web page displaying the lifestyle improvement specific measure information. The item “ABC Gym” and the item “DEF Service” shown in FIG. 6 are the displayed lifestyle improvement specific measure information. The user can select each of the displayed items.

The lifestyle improvement specific measure presenting unit 106 presents more specific details of the lifestyle improvement specific measure information selected by the user from the list of the lifestyle improvement specific measure information presented by the lifestyle improvement specific measure list generating unit 103.

FIG. 7 is an explanatory diagram showing an example of displaying of one lifestyle improvement specific measure information. FIG. 7 shows an example of the case where the item “ABC Gym” is selected from the list of the lifestyle improvement specific measure information shown in FIG. 6. FIG. 7 also shows an example of a web page displaying the lifestyle improvement specific measure information.

In the example shown in FIG. 7, the contents of the “Details” section of the lifestyle improvement specific measure information are also displayed. When the button “View Details” shown in FIG. 7 is clicked, for example, the home page of the company that provides the ABC Gym or a web page where the user can register as a member of the ABC Gym is displayed. As described above, the lifestyle improvement specific measure presenting unit 106 can present more specific details of the lifestyle improvement specific measure information selected by the user.

With the above configuration, the lifestyle improvement specific measure presenting device 100 can present specific measures for lifestyle improvement that is compatible with the lifestyle improvement strategy set by the user. Users who follow the presented specific measures for lifestyle improvement can work on improving his/her lifestyle with high motivation

[Description of Operation]

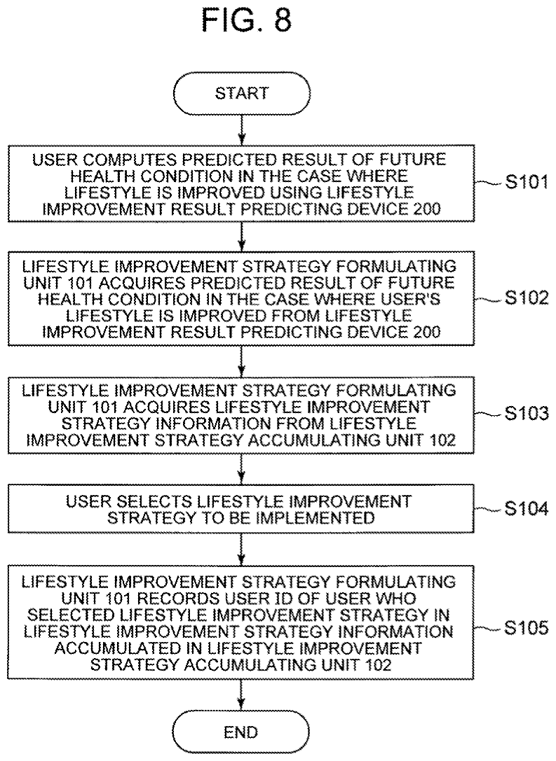

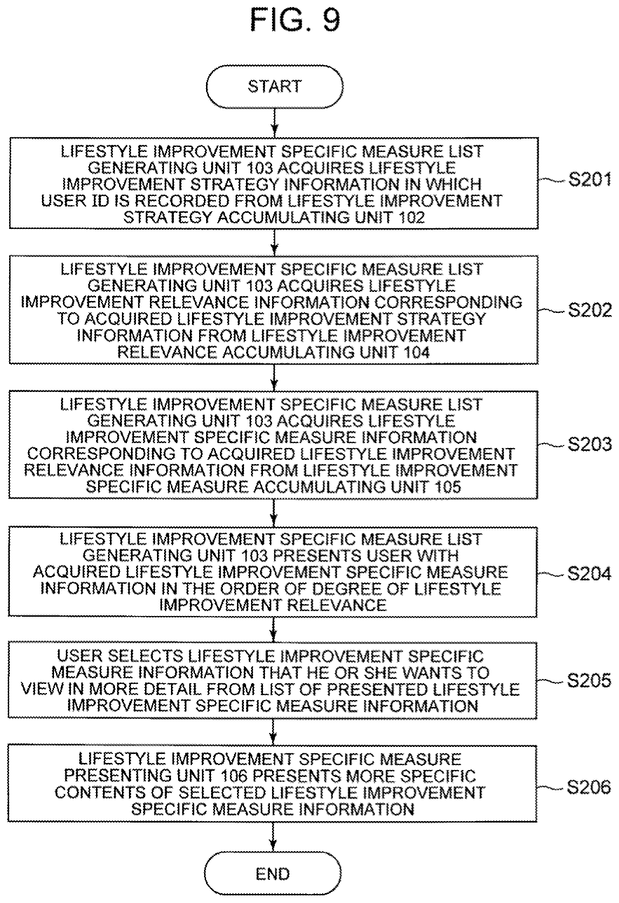

The following describes the operation of the lifestyle improvement specific measure presenting device 100 of this example embodiment with reference to FIGS. 8 and 9.

First, the operation of the lifestyle improvement strategy formulating unit 101 of the lifestyle improvement specific measure presenting device 100 of this example embodiment formulates a lifestyle improvement strategy will be described with reference to FIG. 8. FIG. 8 is a flowchart showing the operation of the lifestyle improvement strategy formulation process by the lifestyle improvement specific measure presenting device 100 of the first example embodiment.

First, the user computes the predicted result of the future health condition in the case where the lifestyle is improved using the lifestyle improvement result predicting device 200 (step S101).

Next, the lifestyle improvement strategy formulating unit 101 acquires the computed predicted result of the future health condition in the case where the user's lifestyle is improved from the lifestyle improvement result predicting device 200 (step S102).

Next, the lifestyle improvement strategy formulating unit 101 acquires the lifestyle improvement strategy information from the lifestyle improvement strategy accumulating unit 102 based on the acquired predicted result (step S103). The lifestyle improvement strategy formulating unit 101 presents the acquired lifestyle improvement strategy information to the user.

Next, the user selects a lifestyle improvement strategy to be implemented from among the lifestyle improvement strategies indicated by the presented lifestyle improvement strategy information (step S104).

Next, the lifestyle improvement strategy formulating unit 101 records the user ID of the user who selected the lifestyle improvement strategy in the lifestyle improvement strategy information including the selected lifestyle improvement strategy accumulated in the lifestyle improvement strategy accumulating unit 102 (step S105). After recording the user ID, the lifestyle improvement strategy formulating unit 101 terminates the lifestyle improvement strategy formulation process.

As in the process of step S104, the user formulates a lifestyle improvement strategy using the lifestyle improvement strategy formulating unit 101 of the lifestyle improvement specific measure presenting device 100. When formulating the lifestyle improvement strategy, the user may receive advice from a doctor, public health nurse, or the like.

Next, the operation of the lifestyle improvement specific measure presenting device 100 of this example embodiment presents the user with specific measures for lifestyle improvement will be described with reference to FIG. 9. FIG. 9 is a flowchart showing the operation of the specific measures for lifestyle improvement presentation process by the lifestyle improvement specific measure presenting device 100 of the first example embodiment.

First, the lifestyle improvement specific measure list generating unit 103 acquires the lifestyle improvement strategy information in which the user's user ID is recorded from the lifestyle improvement strategy accumulating unit 102 (step S201).

Next, the lifestyle improvement specific measure list generating unit 103 acquires the lifestyle improvement relevance information corresponding to the lifestyle improvement strategy information acquired in step S201 from the lifestyle improvement relevance accumulating unit 104 (step S202). Specifically, the lifestyle improvement specific measure list generating unit 103 acquires the lifestyle improvement relevance information including the item number of the acquired lifestyle improvement strategy information.

Next, the lifestyle improvement specific measure list generating unit 103 acquires the lifestyle improvement specific measure information corresponding to the lifestyle improvement relevance information acquired in step S202 from the lifestyle improvement specific measure accumulating unit 105 (step S203). Specifically, the lifestyle improvement specific measure list generating unit 103 acquires the lifestyle improvement specific measure information including the item number of the acquired lifestyle improvement relevance information.

By executing the processes of step S201 to step S203, the lifestyle improvement specific measure presenting device 100 can map the lifestyle improvement strategy formulated by the user to the specific measures for lifestyle improvement registered in the health checkup result prediction simulation system 10.

Next, the lifestyle improvement specific measure list generating unit 103 presents the user with the acquired lifestyle improvement specific measure information in the order of the degree of lifestyle improvement relevance to the formulated lifestyle improvement strategy (step S204). Note that the lifestyle improvement specific measure list generating unit 103 may present the lifestyle improvement specific measure information in any order other than the order of the degree of lifestyle improvement relevance.

By executing the processes up to and including step S204, the lifestyle improvement specific measure presenting device 100 can present the user with a list of lifestyle improvement specific measure information. In addition, the lifestyle improvement specific measure presenting device 100 can present the lifestyle improvement specific measure information that has a high degree of lifestyle improvement relevance to the lifestyle improvement strategy as priority, i.e., the lifestyle improvement specific measure information that is more suitable for the user as priority.

Next, the user selects the lifestyle improvement specific measure information that he or she wants to view in more detail from the list of presented lifestyle improvement specific measure information (step S205).

Next, the lifestyle improvement specific measure presenting unit 106 acquires the more specific details of the selected lifestyle improvement specific measure information from the lifestyle improvement specific measure accumulating unit 105. Then, the lifestyle improvement specific measure presenting unit 106 presents the user with the acquired contents (step S206). After the presentation, the lifestyle improvement specific measure presenting device 100 terminates the specific measures for lifestyle improvement presentation process.

### EFFECTS OF INVENTION

The lifestyle improvement strategy formulating unit 101 of the lifestyle improvement specific measure presenting device 100 of this example embodiment can formulate a lifestyle improvement strategy based on the predicted results of the future health condition in the case where the user's lifestyle is improved as predicted by the lifestyle improvement result predicting device 200. In other words, the lifestyle improvement strategy formulating unit 101 can formulate a lifestyle improvement strategy that is compatible with the lifestyle habit of the user who is required to improve.

In addition, the lifestyle improvement specific measure list generating unit 103 of the lifestyle improvement specific measure presenting device 100 can present specific measures for lifestyle improvement that is compatible with the user's lifestyle improvement strategy that has been formulated. In other words, the lifestyle improvement specific measure list generating unit 103 can present specific measures for lifestyle improvement that are more related to the lifestyle habit of the user who is required to improve.

As described above, the lifestyle improvement specific measure presenting device 100 of this example embodiment can present specific measures for lifestyle improving that is compatible with the lifestyle improvement strategy set by the user. The user can work on improving his/her lifestyle with high motivation by following the presented specific measures for lifestyle improving.

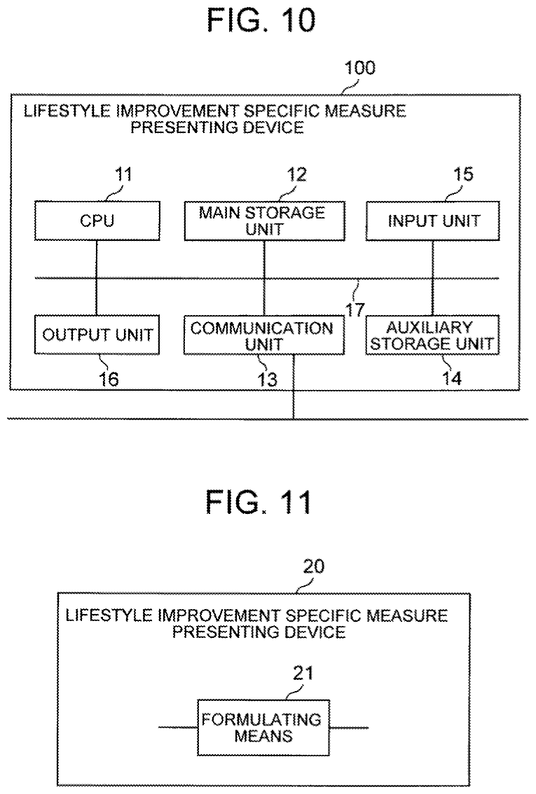

A specific example of a hardware configuration of the lifestyle improvement specific measure presenting device 100 according to the present example embodiment will be described below. FIG. 10 is an explanatory diagram showing an example of a hardware configuration of the lifestyle improvement specific measure presenting device according to the present invention.

The lifestyle improvement specific measure presenting device 100 shown in FIG. 10 includes a CPU (Central Processing Unit) 11, a main storage unit 12, a communication unit 13, and an auxiliary storage unit 14. The lifestyle improvement specific measure presenting device 100 also includes an input unit 15 for the user to operate and an output unit 16 for presenting a processing result or a progress of the processing contents to the user.

The lifestyle improvement specific measure presenting device 100 is realized by software, with the CPU 11 shown in FIG. 10 executing a program that provides a function that each component has.

Specifically, each function is realized by software as the CPU 11 loads the program stored in the auxiliary storage unit 14 into the main storage unit 12 and executes it to control the operation of the lifestyle improvement specific measure presenting device 100.

The lifestyle improvement specific measure presenting device 100 shown in FIG. 10 may include a DSP (Digital Signal Processor) instead of the CPU 11. Alternatively, the lifestyle improvement specific measure presenting device 100 shown in FIG. 10 may include both the CPU 11 and the DSP.

The main storage unit 12 is used as a work area for data and a temporary save area for data. The main storage unit 12 is, for example, RAM (Random Access Memory). The lifestyle improvement strategy accumulating unit 102, the lifestyle improvement relevance accumulating unit 104, and the lifestyle improvement specific measure accumulating unit 105 are realized by the main storage unit 12.

The communication unit 13 has a function of inputting and outputting data to and from peripheral devices through a wired network or a wireless network (information communication network).

The auxiliary storage unit 14 is a non-transitory tangible medium. Examples of non-transitory tangible media are, for example, a magnetic disk, an optical magnetic disk, a CD-ROM (Compact Disk Read Only Memory), a DVD-ROM (Digital Versatile Disk Read Only Memory), a semiconductor memory.

The input unit 15 has a function of inputting data and processing instructions. The input unit 15 is, for example, an input device such as a keyboard or a mouse.

The output unit 16 has a function to output data. The output unit 16 is, for example, a display device such as a liquid crystal display device, or a printing device such as a printer.

As shown in FIG. 10, in the lifestyle improvement specific measure presenting device 100, each component is connected to the system bus 17.

The auxiliary storage unit 14 stores programs for realizing the lifestyle improvement strategy formulating unit 101, the lifestyle improvement specific measure list generating unit 103, and the lifestyle improvement specific measure presenting unit 106 in the first example embodiment.

The lifestyle improvement specific measure presenting device 100 may be implemented with a circuit that contains hardware components inside such as an LSI (Large Scale Integration) that realize the functions shown in FIG. 2, for example.

The lifestyle improvement specific measure presenting device 100 may be realized by hardware that does not include computer functions using elements such as a CPU. For example, some or all of the components may be realized by a general-purpose circuit (circuitry) or a dedicated circuit, a processor, or a combination of these. They may be configured by a single chip (for example, the LSI described above) or by multiple chips connected via a bus. Some or all of the components may be realized by a combination of the above-mentioned circuit, etc. and a program.

In the case where some or all of the components are realized by a plurality of information processing devices, circuits, or the like, the plurality of information processing devices, circuits, or the like may be centrally located or distributed. For example, the information processing devices, circuits, etc. may be realized as a client-server system, a cloud computing system, etc., each of which is connected via a communication network.

Next, an overview of the present invention will be explained. FIG. 11 is a block diagram showing an overview of the lifestyle improvement specific measure presenting device according to the present invention. The lifestyle improvement specific measure presenting device 20 according to the present invention includes a formulating means 21 (for example, the lifestyle improvement strategy formulating unit 101) which formulates a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

With such a configuration, the lifestyle improvement specific measure presenting device can present specific measures for lifestyle improvement which users can work on with high motivation.

The lifestyle improvement specific measure presenting device 20 may include a first storage means (for example, the lifestyle improvement specific measure accumulating unit 105) which stores specific measures for improving lifestyle habits, and an acquisition means (for example, the lifestyle improvement specific measure list generating unit 103) which acquires the specific measures related to the formulated lifestyle improvement strategy from the first storage means.

With such a configuration, the lifestyle improvement specific measure presenting device can present specific measures for lifestyle improvement which users can work on with high motivation.

The formulating means 21 may present the user with one or more formulated lifestyle improvement strategies, and the acquisition means may acquire the specific measures related to the lifestyle improvement strategy selected by the user among the one or more lifestyle improvement strategies.

With such a configuration, the lifestyle improvement specific measure presenting device can present specific measures for lifestyle improvement which users can work on with higher motivation.

The acquisition means may present the user with one or more acquired specific measures, and accept the specific measure selected by the user among the one or more specific measures.

With such a configuration, the lifestyle improvement specific measure presenting device can present specific measures for lifestyle improvement which users can work on with higher motivation.

The lifestyle improvement specific measure presenting device 20 may include a second storage means (for example, the lifestyle improvement relevance accumulating unit 104) which stores relevance information, which is information that includes the lifestyle improvement strategy, the specific measure, and the degree to which the lifestyle improvement strategy and the specific measure is related, and the acquisition means may acquire the specific measures using the relevance information.

With such a configuration, the lifestyle improvement specific measure presenting device can more reliably acquire specific measures related to the formulated lifestyle improvement strategy.

The acquisition means may present the one or more specific measures in the order of their degree of relevance to the formulated lifestyle improvement strategy.

With such a configuration, the lifestyle improvement specific measure presenting device can preferentially present specific measures for lifestyle improvement which users can work on with higher motivation.

The lifestyle improvement specific measure presenting device 20 may include a presenting means which presents the user with a specific content of the accepted specific measure.

The first storage means may store the specific measure together with the specific content of the specific measure, and the presenting means may acquire the specific content from the first storage means.

With such a configuration, the lifestyle improvement specific measure presenting device can present the specific content of specific measures for lifestyle improvement.

The formulating means 21 may formulate the lifestyle improvement strategy on the basis of the predicted result output by a predicting device (for example, the lifestyle improvement result predicting device 200) which predicts the user's future health condition.

With such a configuration, the lifestyle improvement specific measure presenting device can work with the lifestyle improvement result predicting device to present specific measures for lifestyle improvement.

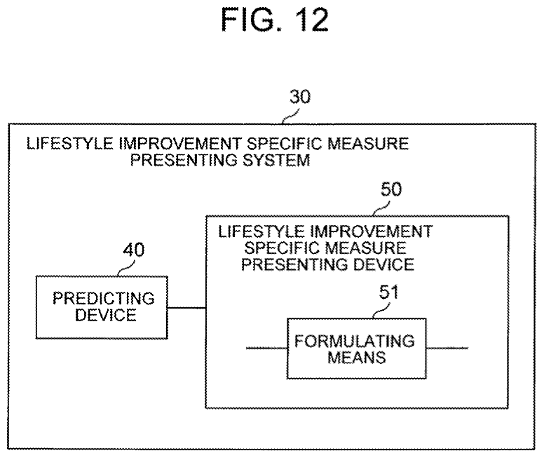

FIG. 12 is a block diagram showing an overview of a lifestyle improvement specific measure presenting system according to the present invention. The lifestyle improvement specific measure presenting system 30 according to the present invention includes a predicting device 40 (for example, the lifestyle improvement result predicting device 200) which predicts a user's future health condition, and a lifestyle improvement specific measure presenting device 50 (for example, the lifestyle improvement specific measure presenting device 100) which includes a formulating means 51 (for example, the lifestyle improvement strategy formulating unit 101) which formulates a lifestyle improvement strategy, which is a behavioral strategy for the user for improving a predetermined lifestyle habit of the user, on the basis of information, which is a predicted result output by the predicting device 40, indicating the predetermined lifestyle habit, included in the predicted result of the future health condition of the user if the predetermined lifestyle habit is improved.

With such a configuration, the lifestyle improvement specific measure presenting system can present specific measures for lifestyle improvement which users can work on with high motivation.

While the present invention has been explained with reference to the example embodiments and examples, the present invention is not limited to the aforementioned example embodiments and examples. Various changes understandable to those skilled in the art within the scope of the present invention can be made to the structures and details of the present invention.

This application claims priority based on Japanese patent application 2019-084865 filed on Apr. 26, 2019, the entire disclosure of which is hereby incorporated herein.

Some or all of the aforementioned example embodiment can be described as supplementary notes mentioned below, but are not limited to the following supplementary notes.

(Supplementary note 1) A lifestyle improvement specific measure presenting device comprising: a formulating means which formulates a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

(Supplementary note 2) The lifestyle improvement specific measure presenting device according to Supplementary note 1, further comprising: a first storage means which stores specific measures for improving lifestyle habits; and an acquisition means which acquires the specific measures related to the formulated lifestyle improvement strategy from the first storage means.

(Supplementary note 3) The lifestyle improvement specific measure presenting device according to Supplementary note 2, wherein the formulating means presents the user with one or more formulated lifestyle improvement strategies, and the acquisition means acquires the specific measures related to the lifestyle improvement strategy selected by the user among the one or more lifestyle improvement strategies.

(Supplementary note 4) The lifestyle improvement specific measure presenting device according to Supplementary note 2 or 3, wherein the acquisition means presents the user with one or more acquired specific measures, and accepts the specific measure selected by the user among the one or more specific measures.

(Supplementary note 5) The lifestyle improvement specific measure presenting device according to Supplementary note 4, further comprising: a second storage means which stores relevance information, which is information that includes the lifestyle improvement strategy, the specific measure, and the degree to which the lifestyle improvement strategy and the specific measure is related, wherein the acquisition means acquires the specific measures using the relevance information.

(Supplementary note 6) The lifestyle improvement specific measure presenting device according to Supplementary note 5, wherein the acquisition means presents the one or more specific measures in the order of their degree of relevance to the formulated lifestyle improvement strategy.

(Supplementary note 7) The lifestyle improvement specific measure presenting device according to any one of Supplementary notes 4 to 6, further comprising: a presenting means which presents the user with a specific content of the accepted specific measure.

(Supplementary note 8) The lifestyle improvement specific measure presenting device according to Supplementary note 7, wherein the first storage means stores the specific measure together with the specific content of the specific measure, and the presenting means acquires the specific content from the first storage means.

(Supplementary note 9) The lifestyle improvement specific measure presenting device according to any one of Supplementary notes 1 to 8, wherein the formulating means formulates the lifestyle improvement strategy on the basis of the predicted result output by a predicting device which predicts the user's future health condition.

(Supplementary note 10) A lifestyle improvement specific measure presenting method comprising: formulating a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

(Supplementary note 11) A computer-readable recording medium recording a lifestyle improvement specific measure presenting program causing, when executed by a computer, the computer to execute: formulating a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

(Supplementary note 12) A lifestyle improvement specific measure presenting system comprising: a predicting device which predicts a user's future health condition; and a lifestyle improvement specific measure presenting device which includes a formulating means which formulates a lifestyle improvement strategy, which is a behavioral strategy for the user for improving a predetermined lifestyle habit of the user, on the basis of information, which is a predicted result output by the predicting device, indicating the predetermined lifestyle habit, included in the predicted result of the future health condition of the user if the predetermined lifestyle habit is improved.

### REFERENCE SIGNS LIST

10 Health checkup result prediction simulation system
11 CPU
12 Main storage unit
13 Communication unit
14 Auxiliary storage unit
15 Input unit
16 Output unit
17 System bus
20, 50, 100 Lifestyle improvement specific measure presenting device
21, 51 Formulating means
30 Lifestyle improvement specific measure presenting system
40 Predicting device
101 Lifestyle improvement strategy formulating unit
102 Lifestyle improvement strategy accumulating unit
103 Lifestyle improvement specific measure list generating unit
104 Lifestyle improvement relevance accumulating unit
105 Lifestyle improvement specific measure accumulating unit
106 Lifestyle improvement specific measure presenting unit
200 Lifestyle improvement result predicting device

What is claimed is:

1. A lifestyle improvement specific measure presenting device comprising:
a memory configured to store instructions; and
a processor configured to execute the instructions to:
formulate a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

2. The lifestyle improvement specific measure presenting device according to claim 1,
wherein the processor is further configured to execute the instructions to:
acquire the specific measures related to the formulated lifestyle improvement strategy from a first storage storing specific measures for improving lifestyle habits.

3. The lifestyle improvement specific measure presenting device according to claim 2,
wherein the processor is further configured to execute the instructions to:
present the user with one or more formulated lifestyle improvement strategies; and
acquire the specific measures related to the lifestyle improvement strategy selected by the user among the one or more lifestyle improvement strategies.

4. The lifestyle improvement specific measure presenting device according to claim 2,
wherein the processor is further configured to execute the instructions to:
present the user with one or more acquired specific measures, and accept the specific measure selected by the user among the one or more specific measures.

5. The lifestyle improvement specific measure presenting device according to claim 4,
wherein the processor is further configured to execute the instructions to:
store relevance information including the lifestyle improvement strategy, the specific measure, and the degree to which the lifestyle improvement strategy and the specific measure is related; and
acquire the specific measures using the relevance information.

6. The lifestyle improvement specific measure presenting device according to claim 5,
wherein the processor is further configured to execute the instructions to:
present the one or more specific measures in the order of their degree of relevance to the formulated lifestyle improvement strategy.

7. The lifestyle improvement specific measure presenting device according to claim 4,
wherein the processor is further configured to execute the instructions to:
present the user with a specific content of the accepted specific measure.

8. The lifestyle improvement specific measure presenting device according to claim 7,
wherein the processor is further configured to execute the instructions to:
acquire the specific content from the first storage storing the specific measure together with the specific content of the specific measure.

9. The lifestyle improvement specific measure presenting device according to claim 1,
wherein the processor is further configured to execute the instructions to:
formulate the lifestyle improvement strategy on the basis of the predicted result output by a predicting device which predicts the user's future health condition.

10. A lifestyle improvement specific measure presenting method comprising:
formulating a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

11. A computer-readable recording medium recording a lifestyle improvement specific measure presenting program causing, when executed by a computer, the computer to execute:
formulating a lifestyle improvement strategy, which is a behavioral strategy for a user for improving a predetermined lifestyle habit of the user, on the basis of information indicating the predetermined lifestyle habit, included in a predicted result of the future health condition of the user if the predetermined lifestyle habit is improved.

12. (canceled)

13. The lifestyle improvement specific measure presenting device according to claim 3,
wherein the processor is further configured to execute the instructions to:
present the user with one or more acquired specific measures, and accept the specific measure selected by the user among the one or more specific measures.

14. The lifestyle improvement specific measure presenting device according to claim 13,
wherein the processor is further configured to execute the instructions to:
store relevance information including the lifestyle improvement strategy, the specific measure, and the degree to which the lifestyle improvement strategy and the specific measure is related; and
acquire the specific measures using the relevance information.

15. The lifestyle improvement specific measure presenting device according to claim 14,
wherein the processor is further configured to execute the instructions to:
present the one or more specific measures in the order of their degree of relevance to the formulated lifestyle improvement strategy.

16. The lifestyle improvement specific measure presenting device according to claim 5,
wherein the processor is further configured to execute the instructions to:
present the user with a specific content of the accepted specific measure.

17. The lifestyle improvement specific measure presenting device according to claim 6,
wherein the processor is further configured to execute the instructions to:
present the user with a specific content of the accepted specific measure.

18. The lifestyle improvement specific measure presenting device according to claim 13,
wherein the processor is further configured to execute the instructions to:
present the user with a specific content of the accepted specific measure.

19. The lifestyle improvement specific measure presenting device according to claim 14,
wherein the processor is further configured to execute the instructions to:
present the user with a specific content of the accepted specific measure.

20. The lifestyle improvement specific measure presenting device according to claim 15,
wherein the processor is further configured to execute the instructions to:
present the user with a specific content of the accepted specific measure.

21. The lifestyle improvement specific measure presenting device according to claim 16,
wherein the processor is further configured to execute the instructions to:
acquire the specific content from the first storage storing the specific measure together with the specific content of the specific measure.
